(19) United States
(12) Patent Application Publication
(10) Pub. No.: US 2009/0157003 A1
(43) Pub. Date: Jun. 18, 2009

(54) Method And Apparatus For Occlusion Prevention And Remediation

(76) Inventors: Daniel W. Jones, Overland Park, KS (US); Amy Childers Lewis, Santa Fe, NM (US)

Correspondence Address: BOYLE FREDRICKSON S.C., 840 North Plankinton Avenue, MILWAUKEE, WI, 53203, US

(21) Appl. No.: 11/956,991

(22) Filed: Dec. 14, 2007

(51) Int. Cl. A61M 5/00

(52) U.S. Cl. 604/131

(57) ABSTRACT

A catheter occlusion prevention or remediation system for use with catheter-based therapeutic or medical liquid delivery to a patient. The system is an adjunct to a catheter liquid delivery arrangement that typically includes an infusion pump hooked to the patient. The system includes a drive that displaces a pulse effector against liquid in a pulse chamber in fluid-flow communication with liquid in the catheter to create a pressure pulse that opens occlusions, breaks away occlusions being formed, and prevents occlusion formation. In one embodiment, the drive charges an accumulator that displaces the pulse effector during a period of low pump motor activity maximizing onboard battery life. In another embodiment, the drive is an actuator with an armature that displaces a plunger into the pulse chamber to form the pulse. The resultant pulses are of sufficient duration and pressure above working pressure to achieve occlusion prevention and remediation.

### FIELD

The present invention is directed to a method and apparatus for prevention and remediation of flow-obstructing catheter occlusions and more particularly to a method and apparatus for proactively doing so by perturbing catheter fluid with increased fluid pressure.

### BACKGROUND

Catheter occlusion occurs when there is a partial or complete obstruction of a catheter which limits or blocks catheter flow. While the causes can be manifold, they can be broken down into two main types of occlusions: thrombotic and non-thrombotic. Thrombotic catheter occlusions are typically caused by deposits of fibrin and blood components that block the subcutaneously-located tip of the catheter. Non-thrombotic catheter occlusions are typically caused by mechanical obstruction, drug precipitation, or lipid residue. It has been estimated that around 60% of all catheter occlusions are thrombotic occlusions.

No matter what the cause, catheter occlusion can be dangerous if not detected and properly treated. Where the occlusion is thrombotic, a thrombolytic agent, such as Tissue Plasminogen Activator (t-PA), can be introduced into the catheter to dissolve the occlusion to restore catheter function. Otherwise, the catheter will need to be replaced.

Where the occlusion is mechanical, quite often the catheter will need to be replaced. For example, where the catheter or its tubing is kinked, where air leaks occur, where the catheter tip is improperly positioned, or where the catheter migrates after placement, the catheter will have to be replaced if attempts to alleviate the problem do not work. However, where occlusion occurs because of a problem with the medication in the catheter precipitating, a medication change or adjustment must also be made. For example, where occlusion due to insulin precipitation occurs, switching to a buffered form of the insulin typically prevents this from happening again.

With the advent of portable or wearable insulin pumps, diabetics have obtained a measure of convenience and freedom not previously known. This has afforded an estimated one million Type 1 diabetics and millions more Type 2 diabetics in the United States the option of using an insulin pump. As such, there are tens of thousands of insulin pumps in use in the United States.

In use, insulin from a reservoir inside the pump is discharged through an infusion set that includes a catheter with a cannula at its free end subcutaneously inserted into the diabetic enabling the discharged insulin to be infused. The amount of insulin along with the rate at which it is discharged from the pump are controlled by a program with diabetic-selected options specifically tailored for the diabetic based on factors such as their weight, blood sugar level and carbohydrate intake. Insulin output can be further increased by the diabetic as needed to output a burst or bolus of insulin such as where it is necessary to introduce a greater amount of insulin right after a meal to supplement the substantially continuous basal rate of insulin delivery that ordinarily is delivered by the pump.

While there have been many insulin pump designs, only a few of them have been successfully commercialized to date. The most common type is a battery-powered, motor driven, insulin infusion pump, such as shown and described in U.S. Pat. No. 4,468,872, U.S. Pat. No. 5,505,709, and U.S. Pat. No. 6,875,195, which typically includes an electric motor coupled to a geared or belted drive train that urges a piston or plunger in a cylinder within the pump in which an syringe-like insulin filled reservoir is received to force insulin from the reservoir into a catheter of a connected infusion set.

Another type of insulin pump that is believed to have never been commercialized is a spring-driven pump described in U.S. Pat. No. 6,736,796, which uses a motor-less pre-pressurized spring-loaded insulin reservoir cartridge whose insulin output is controlled by a micro-electric piezoelectric flow control valve. During operation, force applied by a spring in the reservoir cartridge against insulin in the reservoir cartridge pre-pressurizes the cartridge such that the force of the spring against the insulin causes insulin to be discharged from the pump when the valve is opened. Since the spring supplies the force to discharge insulin from the reservoir cartridge, no electric motor is used.

As previously mentioned, insulin discharged from the insulin reservoir of the pump travels through flexible plastic tubing of a catheter of an infusion set that includes a cannula that has its tip inserted underneath the skin, typically in the region of the abdomen, of the diabetic. Modern infusion sets are equipped with a cannula anchor that usually accommodates an insertion gage used to insert at least part of the cannula underneath the skin of the diabetic. The anchor adhesively attaches to the diabetic to help prevent inadvertent removal or movement of the cannula. The gage typically includes a needle or trocar used to penetrate the skin and place the cannula. After insertion, the needle or trocar is typically removed along with the gage.

Because of the need to make an insulin pump as small and lightweight as possible in order to make it comfortable to wear, considerable effort has been made to reduce weight and bulk in the design process. For example, only a single AA battery, AAA battery, or a very compact custom battery is typically used to power the insulin pump motor and concentrated insulin is used to further reduce size and weight. As a result, current draw on the motor must be low to optimize battery life that insulin is delivered via the catheter at relatively low pressures that are typically less than one psig.

During operation after insertion of the cannula, insulin is supplied by the insulin pump to the diabetic at a relatively low basal rate that is determined based on the diabetic's baseline need for insulin in the absence of carbohydrate intake. Thereafter, in response to increases in blood sugar level, such as what typically occurs after a meal, the diabetic will program the pump to deliver a much larger bolus dose of insulin to metabolize the greater amounts of resultant carbohydrates. While there are many different bolus shapes that can typically be selected or programmed into an insulin pump, all of them involve delivering a rate of insulin flow that is greater than the basal rate over a relatively short period of time ranging anywhere from as little as, for example, fifteen seconds to as long as a minute or two either in anticipation of or in reaction to increased carbohydrate intake. Despite the increased insulin flow rate resulting from a bolus dose, the pressure of the insulin flowing through the catheter to the diabetic patient usually remains less than one psig.

Despite such relatively low insulin flow rates and such low insulin flow volumes, occlusion of infusion sets can and does occur frequently enough to be of serious concern to a diabetic using an insulin pump. In fact, the incidence of catheter occlusions in pediatric diabetics is significantly greater than in adults, possibly because insulin flow rates and volumes used in pediatric insulin pumps are even less than for adults.

When an occlusion occurs, insulin flow into the diabetic is blocked, which can cause blood sugar levels to rise to dangerous levels. If blood sugar levels get too high, a condition known as hyperglycemia can occur. Should blood sugar levels remain too high for too long as a result of an undetected occlusion, ketoacidosis can occur which can in extreme cases lead to coma and even death.

Since an insulin pump is essentially an open loop system that is not capable of detecting occlusions on its own, the pump continues to discharge insulin into the catheter when there is an occlusion. Where a diabetic has programmed the pump to deliver a bolus of insulin that fails to lower blood sugar due to an occlusion, it is not unusual for the diabetic to program the pump to deliver another insulin bolus. Should the resultant buildup of insulin upstream of the catheter cause an increase in pressure that “blows out” the occlusion, an excessive onrush of insulin entering the body can unintentionally cause blood sugar levels to drop precipitously low causing an equally dangerous condition known as hypoglycemia to occur. If blood sugar levels get too low, hypoglycemia can also lead to a coma and even death.

Even where an occlusion is detected in time to avoid these extreme conditions from occurring, the swings in blood sugar which are almost certain to occur cause damage over time. Such undesired excessive variability in blood sugar levels that can occur over time are unhealthy because it can accelerate the occurrence of diabetes related complications that include nerve, eye, kidney, heart and blood vessel damage.

While attempts have been made to monitor insulin pressure or torque readings from part of the insulin pump drive train, such as is disclosed in U.S. Pat. No. 6,659,980 and U.S. Pat. No. 6,656,148, in an attempt to detect an occlusion and notify the diabetic with an alarm should any reading exceed a predetermined threshold, it is believed that to date none of these arrangements are capable of ensuring adequate insulin flow while still reliably, consistently and/or repeatably maintaining alarm protocols. And even if an occlusion is detected, these arrangements do nothing to prevent or remediate the occlusion. At best, the alarm notifies the diabetic of a likely occlusion requiring replacement of the occluded infusion set with a new one.

Previously discussed U.S. Pat. No. 6,736,796 discloses an alarm that is triggered by a pair of pressure sensors located at two different places along a labyrinth insulin flow passage in the pump when their pressure sensor readings differ from indicating a pressure differential, indicative of normal insulin flow, to having equal pressures, indicative of blocked flow. However, before triggering the alarm when it is first detected that the pressure readings are the same, the '796 patent discloses first opening the piezoelectric flow control valve to allow insulin flow to open the blockage. If that does not cause differential pressure between the two pressure sensors to be reestablished then the alarm is triggered.

The arrangement and method disclosed in the '796 patent suffers from a number of inherent drawbacks, not the least of which is its apparent lack of any commercial success whatsoever. First, since insulin pumps by their very nature typically achieve relatively low working pressures of less than one psig during operation due to size constraints and because the insulin is so concentrated, sensing differential pressures is not a reliable way to detect blockages because the pressure difference will typically not vary a great deal between the two pressure sensors because the working pressure is already so low. Second, because the line or lumen of the catheter downstream of the pump is made of a relatively compliant material, like PVC, any increase in pressure created by opening the flow control valve will cause the line or lumen to expand or “give” somewhat increasing the available volume into which insulin under greater pressure can occupy upstream of any blockage. Thus, the resultant insulin flow can reestablish the desired pressure differential without opening the blockage. Finally, separate and independent of these drawbacks is the fact that opening the flow control valve for no more than a few milliseconds as disclosed in the '796 patent simply will not increase pressure enough, if at all, in the catheter line to have any impact on any blockage that has occurred.

What is needed is an apparatus and method for preventing occlusions from occurring as well as preventing those occlusions that begin to form from significantly blocking insulin flow through occlusion remediation that reduces or eliminates the occlusion. What is also needed is such an apparatus and method that can be retrofitted to existing insulin pumps. What is still further needed is such an apparatus and method that is compatible with such occlusion detection arrangements.

### SUMMARY

The present invention provides a method and apparatus for prevention or remediation of catheter inclusions by providing a catheter liquid pressurization system downstream of the source of liquid and upstream of the patient that provides one or more pressure pulses or spikes to open up existing occlusions, to break up occlusions in the process of forming, and to proactively prevent the formation of new occlusions. The catheter liquid pressurization system preferably is a secondary system that can be located between any primary catheter liquid delivery system, like an infusion pump, and the patient with the catheter liquid pressurization system being configured to direct each pressure pulse it produces toward the subcutaneous catheter-tissue interface in the patient where occlusions tend to form.

In a preferred embodiment, the catheter liquid delivery system is a medication infusion pump that preferably is an insulin pump. The catheter liquid pressurization system is a secondary system located between the primary pumping system of the insulin pump and a diabetic patient being infused with insulin from the pump. To help optimize the magnitude of the pressure pulse that reaches the catheter-tissue interface, there preferably is a one-way flow valve disposed between the primary and secondary systems such that any pressure pulse created by the secondary pressurization system will only flow towards the catheter-tissue interface. Such a catheter liquid pressurization system advantageously can be integrally incorporated into the pump, can be configured as a separate unit that is retrofitted to an existing pump, or can be configured as a standalone unit that is inserted in a catheter line.

Where used with an insulin pump, the insulin pump preferably has an insulin pumping system that employs an electric motor as a drive that cooperates with a pump assembly which uses a plunger driven by the motor to discharge insulin from a reservoir cartridge within the pump. Insulin discharged from the reservoir cartridge passes through the one-way valve of the secondary pressurization system where it flows through a catheter line of an infusion set to a cannula subcutaneously inserted into peritoneal tissue of a diabetic patient.

The secondary pressurization system has a pressure pulse generator that includes a pulse chamber downstream of the one-way valve in which a pressure pulse is formed during pulse generator operation. A movable pulse effector interacts with insulin in the pulse chamber during operation to rapidly displace liquid in the chamber creating a pressure pulse or spike having a pressure greater than the working pressure. The pulse pressure preferably is high enough to ensure the pressure pulse travels along the full length of the catheter line to the catheter-tissue interface with enough force to open any occlusion in the way, break up any occlusion being formed, as well as prevent the formation of any occlusion.

The pulse generator includes a drive arrangement that cooperates with the pulse effector to drive the pulse effector into the pulse chamber to form a suitably high pressure pulse. The drive arrangement includes a prime mover that can be and preferably is electrically powered. Preferred prime movers include an electric motor or an electromagnetic actuator.

In one preferred embodiment, the pulse generator utilizes an electric motor drive that is coupled to an energy accumulator that is in turn coupled to a reciprocating pulse effector that has an arm from which a pulse producing head extends. The head is disposed in a pulse chamber formed of a substantially rigid outer casing in which a collapsible, flexible insulin filled bladder is received.

During operation, the pulse generator electric motor is operated to charge the accumulator so that when the accumulator is triggered it will rapidly displace the head of the pulse effector into the pulse chamber casing and against the bladder causing a pressure pulse of insulin to be discharged from the pulse chamber into the catheter toward the catheter-tissue interface. To optimize insulin pump battery life, the pulse generator electric motor preferably is operated only while the insulin pump motor is delivering basal insulin flow to the patient. To help maximize the effect of the pressure pulse, the accumulator is triggered only during delivery of an insulin bolus. Preferably, the accumulator stores enough potential energy to mechanically drive the pulse effector head at least a plurality of times during the bolus such that there are at least a plurality pressure pulses outputted during the bolus.

In another preferred embodiment, the drive is a linear actuator that has a reciprocating armature that extends outwardly to drive a plunger into the pulse chamber to displace insulin from the pulse chamber creating a pressure pulse. While the linear actuator can be energized to produce a pressure pulse during a bolus, it is also preferable to energize the linear actuator during basal insulin delivery to minimize the combined current draw of the linear actuator and the insulin pump motor.

In a preferred method of operation, each pressure pulse lasts between one quarter of a second and two seconds to help ensure it has sufficient duration to achieve the desired occlusion prevention and remediation. In a preferred implementation of the method, each pressure pulse lasts between three quarters of a second and 1½ seconds for this purpose.

To ensure the pressure pulse is of sufficient magnitude to achieve the desired occlusion prevention and remediation, each pressure pulse preferably also has a pressure that is at least 1½ times the working pressure of the liquid in the catheter. In another preferred implementation, each pressure pulse preferably has a pressure that is at least two times working pressure. In still another preferred implementation, each pressure pulse has a pressure of at least three times working pressure. In a further implementation, each pressure pulse has a pressure of at least five times working pressure.

Various features and advantages of the present invention will also be made apparent from the following detailed description and the drawings.

### DRAWING DESCRIPTION

Preferred exemplary embodiments of the invention are illustrated in the accompanying drawings in which like reference numerals represent like parts throughout and in which:

FIG. 1 is a longitudinal cross section view of a therapeutic or medical liquid delivery device equipped with a first preferred embodiment of a secondary pressurization system for prevention and remediation of occlusions;

FIG. 2 is a fragmentary front transverse cross sectional view of the secondary pressurization system of FIG. 1.

FIG. 3 is an exploded longitudinal cross sectional view of the secondary pressurization system of FIG. 1 adapted for retrofit to a therapeutic or medical liquid delivery device;

FIG. 4 is cross section view of the secondary pressurization system of FIG. 1 adapted for standalone use;

FIG. 5 is a longitudinal cross section view of a therapeutic or medical liquid delivery device equipped with a second preferred embodiment of a secondary pressurization system for prevention and remediation of occlusions;

FIG. 6 is a fragmentary front transverse cross sectional view of the secondary pressurization system of FIG. 5;

FIG. 7 is a graph showing basal insulin flow delivery operation of an insulin pump;

FIG. 8 is a second graph depicting insulin bolus operation of the insulin pump;

FIG. 9 is a graph showing occlusion prevention and remediation perturbations in flow during basal insulin flow delivery; and

FIG. 10 is a graph showing occlusion prevention and remediation perturbations in flow during delivery of a bolus as well as during basal flow.

Before explaining embodiments of the invention in detail, it is to be understood that the invention is not limited in its application to the details of construction and the arrangement of the components set forth in the following description or illustrated in the drawings. The invention is capable of other embodiments or being practiced or carried out in various ways. Also, it is to be understood that the phraseology and terminology employed herein is for the purpose of description and should not be regarded as limiting.

### DETAILED DESCRIPTION

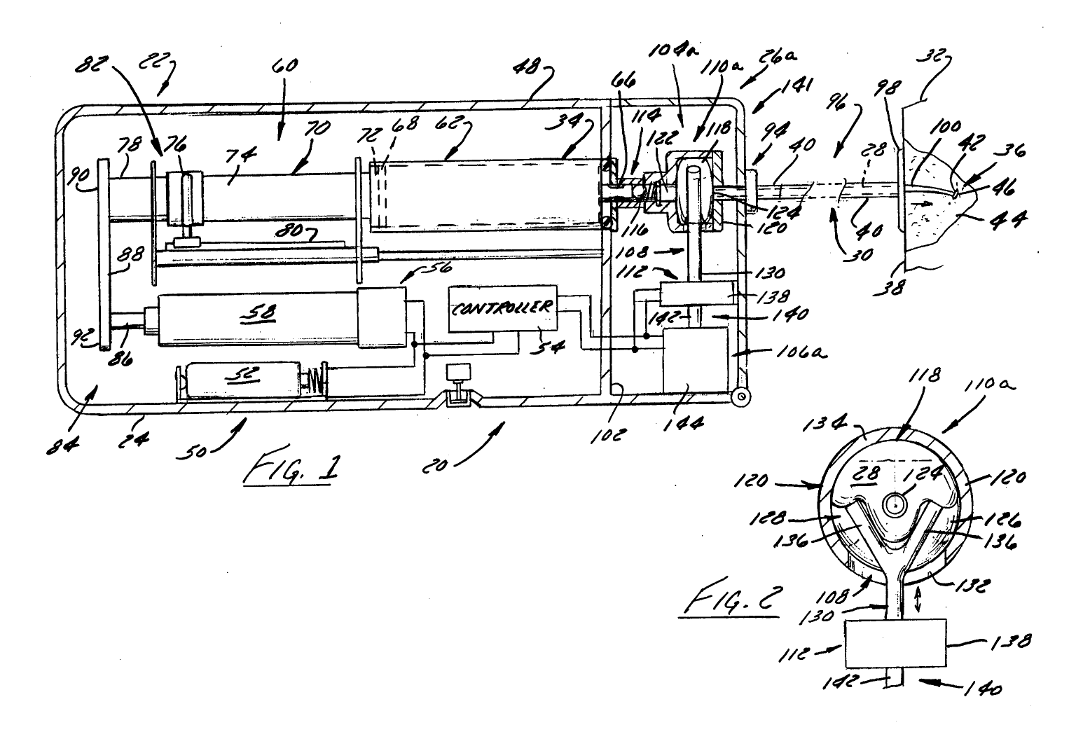

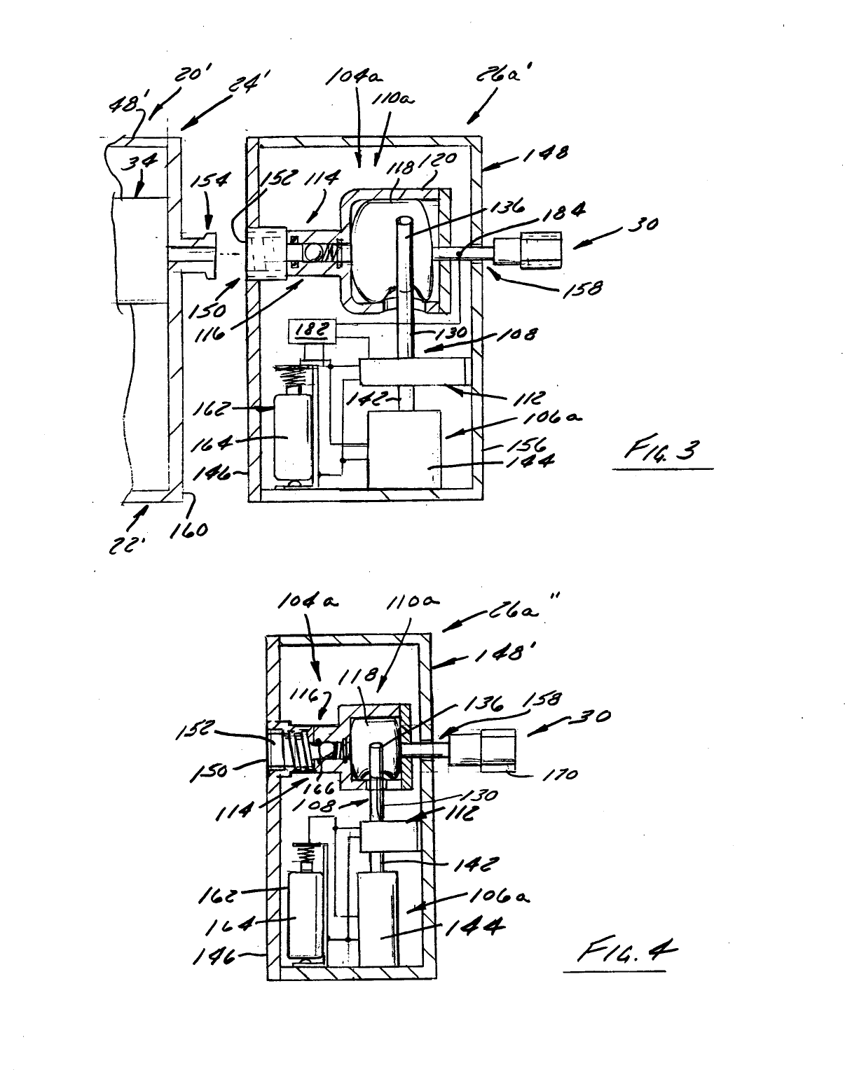

FIG. 1 illustrates a preferred embodiment of a medication delivery device 20 that is an infusion pump 22, preferably an insulin pump 24, which is equipped with an occlusion prevention or remediation apparatus 26a that perturbs liquid 28, such as insulin, introduced intravenously via catheter 30 into a patient 32 (only a portion of whom is shown in FIG. 1), such as a diabetic or the like. Such an occlusion prevention or remediation apparatus 26a can be an integral part of the pump 24, such as is depicted in FIG. 1, or can be constructed as a stand alone module used independently of or as a retrofit to existing infusion pumps, such as is shown in FIGS. 3 and 4. Such an occlusion prevention or remediation apparatus 26a perturbs liquid 28 that has been discharged from a cartridge 34 in the pump 24 thereby enabling occlusion preventing or occlusion remediation perturbations in catheter fluid pressure to be communicated through liquid 28 in the catheter 30 to an occlusion site that is typically located below the skin 38 of the patient 32 and at or upstream of a subcutaneous catheter-tissue interface 36. In the embodiment shown in FIG. 1, an occlusion 46 is shown at the catheter-tissue interface 36 where the end of the catheter tip 42 discharges liquid 28 into tissue 44 of the patient 32.

In a method of the prevention or remediation of catheter occlusions, an occlusion prevention or remediation apparatus 26a constructed in accordance with the present invention perturbs liquid 28 in a line 40 of the catheter 30 so as to rapidly raise fluid pressure high enough above the downstream catheter fluid pressure such that a pressure wave, pulse or spike travels through the liquid 28 in the catheter 30 to the subcutaneous catheter-tissue interface 36 located where the tip 42 of the catheter 30 discharges liquid 28 into tissue 44 of the patient 32, e.g. diabetic. These catheter line pressure perturbations not only prevent formation of such a catheter occlusion, like occlusion 46, but also preferably remediate existing occlusions or occlusions in the process of being formed by proactively breaking them up. Catheter line pressure perturbations are repeated over time to further ensure any occlusions in formation that were broken up by one or more prior pressure perturbations remain broken up and to prevent the formation of new occlusions. Such an occlusion prevention or remediation apparatus 26a is advantageously versatile because it is effective not only against thrombotic occlusions, which are typically caused by fibrin and/or blood components clustering at and around the catheter-tissue interface 36, but can also be effective against precipitation type occlusions, such as where medication in the liquid 28 in the catheter precipitates out of solution.

With continued reference to FIG. 1, the pump 24 is an insulin pump that has a housing 48 that typically is made of plastic and that preferably is fluid-tight in construction. Within the housing 48 is a power source 50 that preferably is a battery 52, such as an AA or AAA cell or the like. As insulin pumps 24 have gotten smaller and lighter so have their batteries such that where an off-the-shelf battery is used it typically consists of a single AA cell 52 that powers the entire pump.

The power source 50 supplies electrical power to a controller 54 that preferably is or includes a processor such that it is programmable. Where equipped with a processor, the processor can be a microprocessor, a microcontroller, an FPGA, or the like. It is contemplated that the power source 50 also supplies electric power to a prime mover 56, such as an electric motor 58, which drives the pump 24 in response to control signals from the programmable controller 54 using power from the battery 52. Where an electric motor, e.g. motor 58, is employed, it is contemplated that the motor be a relatively low speed, low current draw electric motor that is designed to provide enough motive power to ensure adequate fluid discharge during pump operation at a sufficiently high enough pressure while minimizing battery drain and maximizing battery life.

The motor 58 is operatively coupled to a pump drive assembly 60 that is constructed and arranged to cause fluid to be discharged from the cartridge 34 ultimately into the line 40 of the catheter 30. In the preferred embodiment shown in FIG. 1, the pump drive assembly 60 communicates with a chamber 62 which has a discharge outlet 66 at one end and which can include a displaceable end wall 68 at its other end. The displaceable end wall 68 can also be formed of or part of the cartridge 34.

The chamber 62 can house, include or be formed of the cartridge 34, which typically is filled with a fluid that is or contains medication or the like used in treating or facilitating treatment of a condition that can be an illness, disease, infection, ailment or the like. Medication refers to any substance that is used to help cure, alleviate or prevent an illness, disease, infection, ailment or the like.

For diabetic applications, the reservoir cartridge 64 is filled with an insulin, such as a fast acting or slow acting insulin. Such insulin typically possesses a potency of anywhere from 40 to 100 units per milliliter, i.e. U-40 to U-100. While the end wall 68 is shown in FIG. 1 as part of the chamber 62 but can be an end wall of the cartridge 34. While a cartridge 34 is shown received in chamber 62, the present invention contemplates an arrangement where no cartridge 34 is used with the chamber 62 serving as a reservoir from which liquid 28 is discharged during operation of the pump 22.

The plunger 70 of the pump drive assembly 60 has a head 72 at one end that engages the displaceable end wall 68, such as by abutting against it. The plunger 70 also includes an elongate, generally cylindrical and hollow shaft 74. In the embodiment shown in FIG. 1, a collar 76 driven by a rotary driveshaft 78 and guided by a slide 80 of a drive carriage 82 moves the plunger shaft 74, and hence the plunger 70, as the driveshaft 78 is rotated by the motor 58.

In a preferred embodiment, the driveshaft 78 is threadably coupled to the plunger 70 such that rotation of the driveshaft 78 displaces the plunger 70, which in turn displaces the end wall 68 towards the discharge outlet 66 discharging liquid from the cartridge 34. In such an arrangement, the driveshaft 78 can be threadably, telescopically received within the plunger 70 or vice versa. In such an arrangement, the collar 76 is carried by the plunger 70 such that it displaces in unison therewith. Other arrangements are contemplated.

No matter what the arrangement, the slide 80 can also be used to provide a scale or other suitable information used to provide feedback to the programmable controller 54 as the collar 76 displaces during cartridge discharge regarding the volume of insulin discharged over time. In the preferred embodiment shown in FIG. 1, the slide 80 is used to provide such feedback.

The driveshaft 78 is operatively coupled by a drivetrain 84 of the pump drive assembly 60 to an output shaft 86 of the motor 58. In the preferred embodiment shown in FIG. 1, the drivetrain 84 includes a belt 88 that extends from a driven sheave or pulley 90 at the end of the driveshaft 78 to a drive sheave or pulley 92 fixed to one end of the motor output shaft 86.

In preparation for operation, the cartridge 34 is filled and placed in the chamber 62 within the pump housing 48. Where the pump 22 is an insulin pump, the cartridge 34 is either filled by the patient 32, i.e. diabetic, with insulin, such as by using a syringe, or is purchased as a pre-filled insulin cartridge before being inserted into the chamber 62. The catheter 30 is subcutaneously inserted into the patient 32 and the catheter line or lumen 40 is connected to a fitting 94, such as a Luer lock fitting or the like, which is in fluid-flow communication with the cartridge 34. Where used to deliver insulin, the catheter 30 can be part of an infusion set 96 that includes an anchor 98 from which a pointed and/or curved cannula 100 extends outwardly subcutaneously into tissue of the patient 32. The line or lumen 40 typically is a length of flexible tubing that runs from the anchor 98 to the fitting 94.

The occlusion prevention or remediation apparatus 26a is located downstream of the source of medication, e.g. cartridge 34, and upstream of the catheter tip 42 or cannula 100. In the preferred embodiment shown in FIG. 1, the occlusion prevention or remediation apparatus 26a is located between the discharge outlet 66 of the pump 22 and the catheter 30.

While the occlusion prevention or remediation apparatus 26a can be constructed as an integral part of the pump 24, such as is depicted in FIG. 1, an occlusion prevention or remediation apparatus 26a′ and 26a″ constructed in accordance with the present invention can also be configured as a standalone unit, such as is depicted in FIGS. 3 and 4, that is either capable of being retrofitted to existing pumps, such as the unit 26a′ shown in FIG. 3, or used just with a catheter line, such as the unit 26a″ shown in FIG. 4. While the occlusion prevention or remediation apparatus 26a is separated by an end wall 102 of the pump housing 48, such an end wall 102 is neither needed nor required for a pump integrally equipped with a prevention or remediation apparatus 26a constructed in accordance with the present invention.

With additional reference to FIG. 2, the occlusion prevention or remediation apparatus 26a includes a pressure pulse generator 104a that includes a drive 106a that is coupled to a pulse effector 108 which cooperates with catheter liquid 28 received in a pressure pulse chamber 110a. The drive 106a preferably is coupled to the pulse effector 108 by an accumulator 112 that stores mechanical energy inputted from the drive 106a in order to build up potential energy that is released when it is desired to produce a perturbation, i.e., a pressure pulse, in liquid 28 in the catheter 30 that is greater than the working pressure of the liquid 28 and which will be of sufficient magnitude such that the pulse travels along the catheter 30 all the way to the catheter-tissue interface 36 where most occlusions tend to occur. Where an accumulator 112 is used, it can be separate from or an integral part of the drive 106a.

To help optimize the magnitude of the pressure pulse, there is a pulse director 114, which in the preferred embodiment shown in FIG. 1 is a one-way valve 114, such as a ball-type check valve, disposed in a coupling or line 116 located between the pump discharge 66 and a bladder 118 of the apparatus 26a that prevents any pressure pulse generated during an occlusion prevention or remediation cycle from entering liquid 28 in the cartridge 34 and dissipating. As a result, each pressure pulse generated during an occlusion prevention or remediation cycle travels at maximum magnitude through the liquid 28 in the catheter line 40 where it most efficiently delivers its occlusion prevention or remediation effects along the line 40 all the way to the subcutaneous tissue interface 36. Such as where a more compact occlusion prevention or remediation apparatus is desired, the coupling or section of line between the discharge 66 of the pump 22 and the bladder 118 of the apparatus 26a may be eliminated.

The bladder 118 of the pressure pulse chamber 10a is compressible and made of a flexible and resilient material, such as PDC or polyolefin, which is received in a casing 120 that is substantially rigid so that the bladder 118 can be compressed by at least part of the effector 108 against the casing 120 to create a pressure pulse in catheter liquid 28 downstream of the cartridge 34. The bladder 118 has an inlet 122 in fluid flow communication with the pump discharge 66 and has an outlet 124 in fluid flow communication with the catheter tube receiving fitting 94. In the preferred embodiment depicted in FIG. 1, the bladder 118 is a compressible pouch formed of a sidewall 126 that can be of endless or substantially endless construction.

While the bladder support casing 120 is annular and preferably generally cylindrical, it can be any suitable shape so long as it substantially rigidly encases the bladder 118 and supports the bladder 118 during pulse creating compression to facilitate pulse creation. The bladder 118 is able to expand as liquid discharged from the cartridge 34 fills it to have a shape that can be substantially complementary to that of the casing 120.

The pulse effector 108 has a pulse generating head 128 disposed in engagement with the bladder sidewall 126 such that rapid displacement of the head 128 against the bladder sidewall 126 compresses the bladder 118 creating a pressure pulse. The head 128 is attached to an arm 130 that extends through an opening 132 in a sidewall 134 of the casing 120 and inside the casing 120. As is best shown in FIG. 2, the head 128 is of forked or V-shaped construction having a pair of tines 136 spaced apart so as to define an acute included angle therebetween. The head 128 preferably is substantially completely received within the bladder support casing 120 with each tine 136 disposed between an inner surface of casing sidewall 134 and an outer surface of the bladder sidewall 126.

The pulse effector arm 130 extends outwardly from the accumulator 112 with the accumulator rapidly displacing the head 128 and arm 130 toward the bladder 118 during pulse creation. Once the accumulator 112 has extended the head 128 as far outwardly as it can go, the accumulator 112 is constructed and arranged to return the head 128 by automatically retracting both the head 128 and arm 130 back to a launch position where the head 128 is fully retracted. Such a retraction arrangement can be of spring-biased construction (not shown) or the like. Once retracted back to the launch position, the head 128 is ready to be extended by the accumulator 112 during another pressure pulse creation cycle.

In one preferred method of occlusion prevention and remediation, the accumulator 112 rapidly displaces the pulse effector head 128 outwardly from the launch position toward and against the bladder 118 to a first pressure pulse creation position disposed a distance away from its fully retracted position and then dwelling a period of time before rapidly displacing the head 128 farther outwardly to a second pressure pulse creation position, such as the fully extended position shown in FIG. 2. In another preferred method of occlusion prevention and remediation, the pressure pulse creation cycle encompasses the accumulator 112 driving the head 128 through a plurality of pairs, i.e., three or more, of pulse creating positions such that at least a plurality of pairs pressure pulses are created.

In one preferred method of operation, the pulse effector head 128 is retracted from its fully extended position shown in FIG. 2 to a fully retracted position (not shown) until it reaches a position where the bladder 118 can nearly completely fill with liquid being discharged from the cartridge 34. For example, in one implementation of such a method of operation, the head 128 is retracted until it is no longer in contact with any part of the bladder sidewall 126 thereby giving the bladder 118 an opportunity to refill. Thereafter, the pulse effector head 128 is urged inwardly from its fully retracted position into the casing 120 until it bears against part of the bladder 118 at an intermediate position between the fully extended position (shown in FIG. 2) and the fully retracted position. As this occurs, a pulse of liquid is forced from the bladder 118 into the catheter line 40 travelling as far as the subcutaneous tissue interface 36 preventing and/or breaking up any occlusion in its path. Depending on the number of intermediate positions, one or more additional pulses can be generated in this manner as the pulse effector 108 is urged further into the casing 120 and against the bladder 118.

In the preferred embodiment shown in FIGS. 1 and 2, the accumulator 112 is a mechanical energy accumulator but can be configured to accept any type of energy input whether such input energy is electrical, mechanical, chemical, etc. so long as the accumulator 112 outputs the stored energy in a mechanical form that drives the effector 108 in a manner that causes pressure pulses to be produced in the liquid 28 in the catheter 30 downstream of the cartridge 34. In a preferred embodiment, the accumulator 112 is a mechanical energy accumulator that accepts rotary or linear input motion from the drive 106a during an energy storage phase of accumulator operation. Such an accumulator 112 preferably has a mechanical energy storage mechanism (not shown) disposed within its housing 138 with its housing 138 fixed to part of the pump, such as the pump housing 48. Where separate from pump 22, the accumulator 112 can be anchored elsewhere, such as to part of a housing 141 of the apparatus 26a.

In a preferred energy storage mechanism embodiment, the energy storage mechanism of the accumulator 112 is a windup energy storage mechanism (not shown) that includes a coil power storage spring (not shown), such as a coil spring of spiral or helical construction, which cooperates with a clutch (not shown), such as a one-way clutch. Such an energy storage mechanism can also include a gear train (not shown) or the like, including a gear train disposed in cooperation with an input shaft that can be a rotary input shaft. An example of a preferred rotary input shaft is an output shaft of an electric motor or the like.

However, where an electric motor is not used to provide mechanical input power, a self-winding mechanical energy storage mechanism can be used that employs the same or like components in combination with a pivotable or rotatable winding mass (not shown), such as a winding rotor or the like, which pivots or rotates during motion of a person wearing the pump walking, turning or otherwise moving around. Where an electric motor is used the charge the accumulator 112, a magnetic-shake generator or wind-up dynamo can be connected to a battery, such as battery 52, used to power the motor to supplement battery power and/or to charge the battery if desired. In a still further embodiment, a wind-up mechanical energy storage mechanism can be employed.

As is shown in FIG. 2, the drive 106a has a coupling 140 that serves as or is otherwise connected to an input of the accumulator 112. In the preferred embodiment shown in FIG. 2, the coupling 140 is an output shaft 142 that serves as or is otherwise connected to an input, e.g. input shaft, of the accumulator 112. In a preferred embodiment, the drive 106a is an electric motor 144 (FIG. 1) that has a rotary output shaft 142 that winds up the mechanical energy storage mechanism (not shown) that is located inside the housing 138 of the accumulator 112.

Although not shown in FIGS. 1 and 2, there is a trigger connected to the pump controller 54 that is activated or energized by the controller 54 to cause the accumulator 112 to discharge its stored energy and drive the pulse effector 108 to produce an occlusion prevention or remediation pressure pulse. Where the apparatus 26a is employed separate from or without any pump, e.g., infusion pump 22, such a controller, e.g. controller 54, can be disposed onboard the apparatus 26a.

In one preferred embodiment, the trigger is an actuator (not shown), such as a linear actuator like a solenoid or voice coil actuator. Such an actuator is disposed onboard the apparatus 26a and can be disposed within the accumulator housing 138 in operable cooperation with the mechanical energy storage mechanism. In another preferred embodiment, the trigger is a piezoelectric actuator (not shown), a rotary actuator, or the like. Other types of triggers and trigger mechanisms can be used.

FIGS. 3 and 4 disclose a discrete embodiment of an occlusion prevention or remediation apparatus 26a′ and 26a″ constructed in accordance with the present invention. The embodiment of the occlusion prevention or remediation apparatus 26a′ shown in FIG. 3 is configured for retrofit attachment to a medication delivery device 20′, such as an insulin infusion pump 24′, which previously lacked any sort of occlusion prevention or remediation device. As such, an occlusion prevention or remediation apparatus 26a′ constructed in accordance with the present invention can be adapted for use with other types of infusion pumps, including those which deliver medication containing liquid intravenously, subcutaneously, arterially, and epidurally. Examples of such suitable infusion pumps include large volume and small volume pumps that can be set up for continuous infusion operation, intermittent infusion operation, and/or patient controlled infusion operation.

To facilitate retrofit attachment to an insulin pump 24′, one sidewall 146 of a housing 148 of the retrofittable occlusion prevention or remediation apparatus 26a′ carries a socket 150, such as female receptacle 152, configured for releasable attachment to a catheter attachment fitting 154, such as a Luer lock fitting or the like, of the pump 24′. So that a catheter 30 of an infusion set, intravenous line, or the like can be attached to the apparatus 26a′, another sidewall 156 of the apparatus housing 148 carries a catheter attachment fitting 158, such as a Luer lock fitting or the like. The apparatus 26a′ preferably has its own onboard power source 162, such as a battery 164 like an AA alkaline battery, an AAA alkaline battery, a lithium battery of similar or same configuration, or another type of suitable power source. If desired, the apparatus 26a′ can also be configured to accept electrical power from a utility power source, such as a source of 120 volt AC power or the like.

At least one other point or means of attachment can be employed to further secure the retrofittable apparatus 26a′ to the housing of the insulin pump, where the apparatus 26a′ is directly attached. For example, an adhesive arrangement (not shown), such as double-sided tape or the like, can be disposed between adjoining sidewalls 146, 160 of the apparatus housing 148 and the pump housing 48′. If desired, a fastener arrangement (also not shown), such as one employing hook and loop fasteners, e.g. VELCRO or the like, can be used, with the fastener arrangement disposed between the respective adjoining sidewalls 146, 160 or in some other suitable fashion. Of course, other methods and arrangements for retrofitting the apparatus 26a′ to such a pump or other medication delivery arrangement can be employed.

FIG. 4 illustrates a standalone occlusion prevention or remediation apparatus 26a″ that is configured for inline catheter use downstream of any source of medication, e.g., medication containing liquid, such as an intravenous bag (not shown), an intravenous pump, or another type of infusion pump, such as one of the pumps discussed above, and upstream of where the catheter 30 is inserted into a patient. Such a catheter can be intravenously, subcutaneously, arterially, or epidurally inserted.

The standalone apparatus 26a″ has a pair of catheter connection fittings 158, 166 with one of the fittings 166 being an inlet fitting that accepts catheter liquid flow from a source of liquid flowing through an attached catheter line 168 and the other one of the fittings 158 being an outlet fitting that enables catheter liquid passing through the apparatus 26a″ to exit the apparatus 26a″ through an attached catheter line 170 that communicates the liquid to a patient. To optimize the magnitude of any pressure pulse produced by the standalone occlusion or remediation apparatus 26a″, a one-way valve 114 is disposed in inlet fitting 166 or slightly downstream of fitting 166. Although not shown in FIG. 4, the standalone apparatus 26a″ has a source of electric power, such as a battery or the like, which can be disposed onboard the apparatus.

With reference once again to FIGS. 1-2, in a method of charging the accumulator 112 where the pump, e.g., insulin pump 24, is battery powered, the accumulator charging motor 144 is run while the pump 24 is discharging liquid from the reservoir at a relatively low flow rate that is lower than a predetermined value, threshold or range. By running the accumulator charging motor 144 when the flow rate is at or below a predetermined value or threshold, the total load placed on the onboard battery 52 by the pump motor 58 and the accumulator charging motor 144 is minimized thereby advantageously extending battery life.

Where the occlusion prevention or remediation apparatus 26a′ or 26a″ is of retrofit or standalone construction, the accumulator trigger can be a controller 182 (FIG. 3) disposed onboard the apparatus 26a′ or 26a″ that is separate from the controller 54 used to control operation of the pumping or infusion device, e.g. insulin pump 24 or 24′ which with the apparatus 26a′ or 26a″ is associated. Such a separate dedicated controller can be coupled to a sensor arrangement 184 (FIG. 3), such as one that includes a fluid pressure sensor or flow measurement device, e.g., flowmeter, that is used to monitor flow characteristics of liquid 28 being discharged from the pumping or infusion device with which the occlusion prevention or remediation apparatus 26a′ or 26a″ is associated. Such a control arrangement that includes such a separate dedicated controller 182 and sensor 184 is used to determine when to trigger the discharge of the accumulator 112 in carrying out an occlusion remediation or prevention cycle in accordance with that discussed in more detail below. Although not shown in FIG. 4, apparatus 26a″ is also equipped with its own controller and sensor arrangement.

In a preferred implementation of a method of charging up the accumulator 112 where the pump 22 is a battery-powered insulin pump, the accumulator charging motor 144 is run while the insulin pump motor 58 is either off or running in a basal flow delivery mode where it is placing a lesser load on the battery 52 (or even no load on the battery 52) such that the combined load on the battery 52 imposed by the accumulator charging motor 144 and the pump motor 58 is within the rated discharge performance curves for the battery used in the pump.

In one preferred implementation, the motors 58, 144 are selected such that their combined power is no greater than 200 milliwatts (mW), assuming constant power performance, basal flow delivery insulin pump motor operation, and accumulator charging motor operation only during basal flow delivery. Where the pump is powered by a single 1.5 volt AA lithium battery, such as an ENERGIZER L91 1.5 volt Lithium battery, a 200 mW combined power draw advantageously ensures a minimum of twenty hours of powered operation.

In another preferred implementation, the motors 58, 144 are selected such that their combined power is no greater than 300 mW, assuming constant power performance, basal flow delivery insulin pump motor operation, and accumulator charging motor operation only during basal flow delivery. Where the pump is powered by a single 1.5 volt AA lithium battery, a 300 mW combined power draw advantageously ensures a minimum of twelve hours of hours of powered operation.

In still another preferred implementation, the motors 58, 144 are selected such that their combined power is no greater than 400 mW, assuming constant power performance, basal flow delivery insulin pump motor operation, and accumulator charging motor operation only during basal flow delivery. Where the pump 24 is powered by a single 1.5 volt AA lithium battery, a 400 mW combined power draw advantageously ensures a minimum of nine hours of hours of powered operation.

In one preferred method implementation, the motors 58, 144 are selected such that their combined power is between 200 mW and 300 mW, assuming constant power performance, basal flow delivery insulin pump motor operation, and accumulator charging motor operation only during basal flow delivery. Where the pump 24 is powered by a single 1.5 volt AA lithium battery, this preferred power operating range advantageously ensures between about twelve and about twenty hours of battery powered operation.

In another preferred method implementation, the motors 58, 144 are selected such that their combined power is between 300 mW and 400 mW, assuming constant power performance, basal flow delivery insulin pump motor operation, and accumulator charging motor operation only during basal flow delivery. Where the pump 24 is powered by a single 1.5 volt AA lithium battery, this preferred power operating range advantageously ensures between about nine and about twelve hours of battery powered operation.

In a preferred method of occlusion clearing pressure pulse cycle operation, the accumulator 112 is triggered driving the pulse effector 108 against the bladder 118 of the pressure pulse chamber 110 during a high catheter liquid flow rate period of operation such that a pressure pulse is delivered during a period of maximum flow and pressure in the catheter 30 to help optimize the ability to open or clear any existing occlusion as well as breakup any occlusion in the process of formation. In one preferred method implementation where the pump 22 is an insulin pump 24, the pressure pulse cycle is performed during an insulin bolus. In one preferred method implementation, a plurality of pressure pulse cycles are performed while a bolus of insulin is being administered to the patient by the pump 24. In another preferred method implementation, at least a plurality of pairs (i.e. at least three) of pressure pulse cycles is performed during an insulin bolus.

Each pressure pulse cycle produces a pressure pulse that preferably has a duration lasting anywhere from 1/100^{th} of a second to as long as two seconds. In one preferred pressure pulse cycle method implementation, each pressure pulse cycle produces a pressure pulse that has a duration lasting anywhere from three quarters of a second to 1½ seconds ensuring that the pressure pulse duration is long enough to open up any occlusion that has formed as well as to clear out any portion of any occlusion that is in the process of being formed.

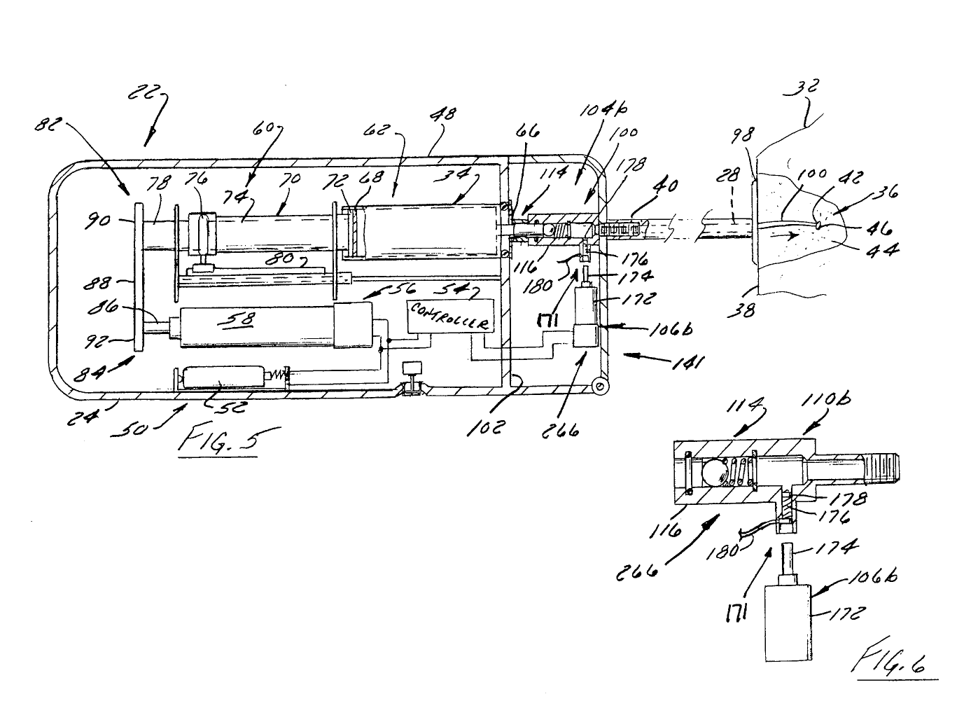

FIGS. 5 and 6 illustrate another preferred embodiment of an occlusion prevention or remediation apparatus 26b constructed in accordance with the present invention that has a pressure pulse generator 104b with a pressure pulse chamber 110b that is of bladderless construction. A reciprocating armature 174 of the drive 106b is part of a pulse effector arrangement 171 discussed in more detail in the following paragraph that cooperates with catheter liquid 28 received in pressure pulse chamber 110b to produce an occlusion remediating or preventing perturbation in the catheter liquid 28.

The drive 106b is a linear motor or linear actuator 172, such as a fast acting solenoid or a voice coil actuator, which has a reciprocating armature 174 that can be extended outwardly against a resiliently biased plunger 176 that is in fluid flow communication with liquid 28 in the chamber 110b. If desired, the armature 174 can be powered or driven in another manner. As the armature 174 is rapidly extended during linear actuator operation, it displaces a diaphragm 178 of the plunger 176 toward and preferably into the chamber 110b. As the diaphragm 178 is displaced toward the chamber 110b, it propels liquid into and out of the chamber 10b creating a pressure pulse that travels along the liquid 28 in the catheter 30 toward the catheter-tissue interface 36. If needed, a back feed line 180 can be provided as shown in FIGS. 5 and 6.

If desired, the armature 174 can be driven by an accumulator (not shown in FIGS. 5 and 6) constructed and operated the same as or like accumulator 112 in a manner like that described above with regard to the preferred embodiment shown in FIGS. 1 and 2. Where an accumulator is used, it can be separate from or an integral part of the drive 106b.

In a preferred method of operation of the occlusion prevention or remediation apparatus 26b where the pump is battery powered, the drive actuator 172 is also actuated during a low catheter liquid flow rate period of operation to conserve overall battery power. In one preferred implementation of a method of operation where the pump is a battery-powered insulin pump, the drive actuator 172 is actuated during a basal flow rate insulin delivery cycle to conserve battery power by operating actuator 172 during a period of low insulin pump motor electrical current demand.

In one preferred implementation, at least a plurality of occlusion clearing pressure pulse cycles are performed by energizing the actuator 172 to extend its armature and hence the plunger to a fully extended pressure pulsing position, causing the actuator to retract its armature along with the plunger, such as by de-energizing the actuator, completing a first pressure pulse cycle and thereafter energizing and de-energizing the actuator 172 again, completing a second pressure pulse cycle. In another preferred implementation, at least a plurality of pairs of discrete and spaced apart pressure pulse cycles are executed in this manner during the basal flow rate insulin delivery cycle.

Each pressure pulse cycle produces a pressure pulse that preferably has a duration lasting anywhere from 1/100^{th} of a second to as long as two seconds. In one preferred pressure pulse cycle method implementation, each pressure pulse cycle produces a pressure pulse that has a duration lasting anywhere from one quarter of a second to 1½ seconds ensuring that the pressure pulse duration is long enough to open up any occlusion that is formed as well as to clear out any portion of any occlusion that is in the process of being formed.

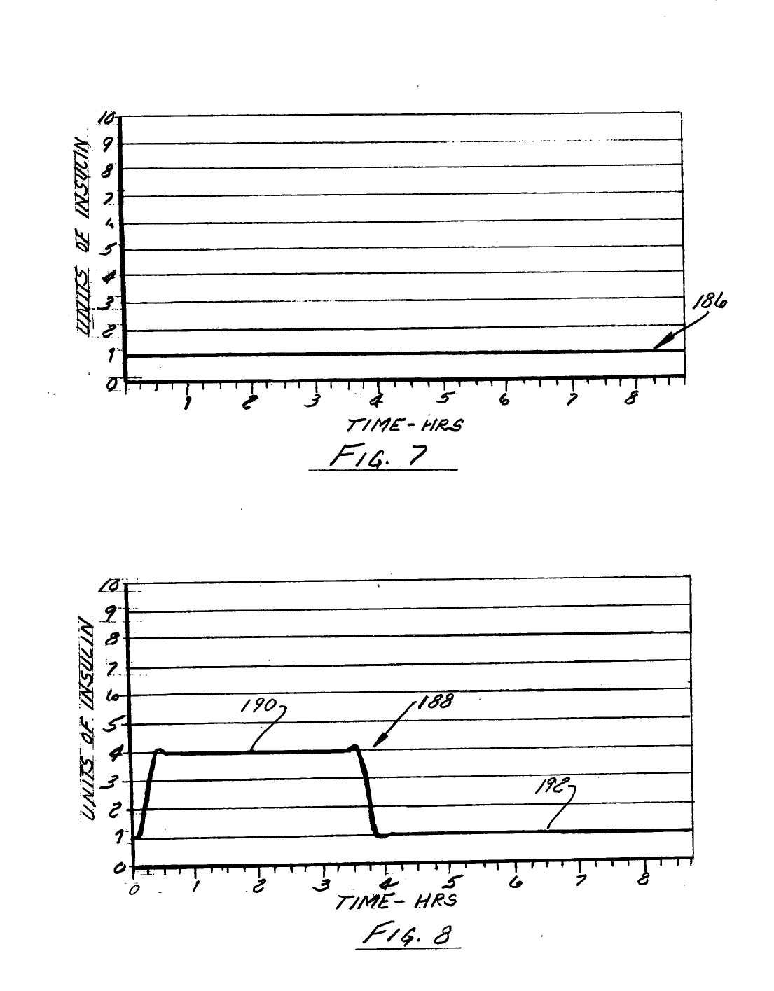

FIGS. 7 and 8 illustrate two typical operational modes of an insulin pump, such as the insulin pump 24 depicted in FIG. 1 that lacks any kind of occlusion remediation apparatus. FIG. 7 illustrates an example of a basal insulin delivery cycle where a relatively low flow rate of insulin, e.g., insulin containing liquid, is discharged from the pump into the patient. As is reflected by the curve 186 shown in the graph of FIG. 7, basal flow is characterized by a steady and relatively low flow rate of insulin. For example, in FIG. 7 a single unit of insulin per hour is discharged all throughout the basal delivery cycle. Typically, basal delivery is employed between meals and while a patient is resting or sleeping.

FIG. 8 illustrates a bolus insulin delivery cycle that is a square wave bolus that lasts for approximately 2½ hours where the insulin flow rate is increased beyond the basal delivery flow rate during that time. Typically, a bolus delivery cycle is manually initiated by the patient in response to eating a meal, ingesting high glycemic foods, or to correct a high blood glucose level. Other types of bolus delivery cycles are possible and include a pre-bolus, a spike bolus, e.g., super bolus, or a combination of a spike and square wave bolus. Where linked to a blood glucose monitor, including a continuous blood glucose monitor, readings from the blood glucose monitor can be used to automatically trigger a bolus insulin delivery cycle or a specific type of bolus insulin delivery cycle.

In the curve 188 shown in the graph of FIG. 8, a bolus portion 190 of the curve indicates that the insulin pump is delivering four units of insulin per hour for a period of approximately 2½ hours. Thereafter, basal delivery resumes such that the remaining portion 192 of the curve is substantially flat or constant at a delivery rate of about one unit per hour.

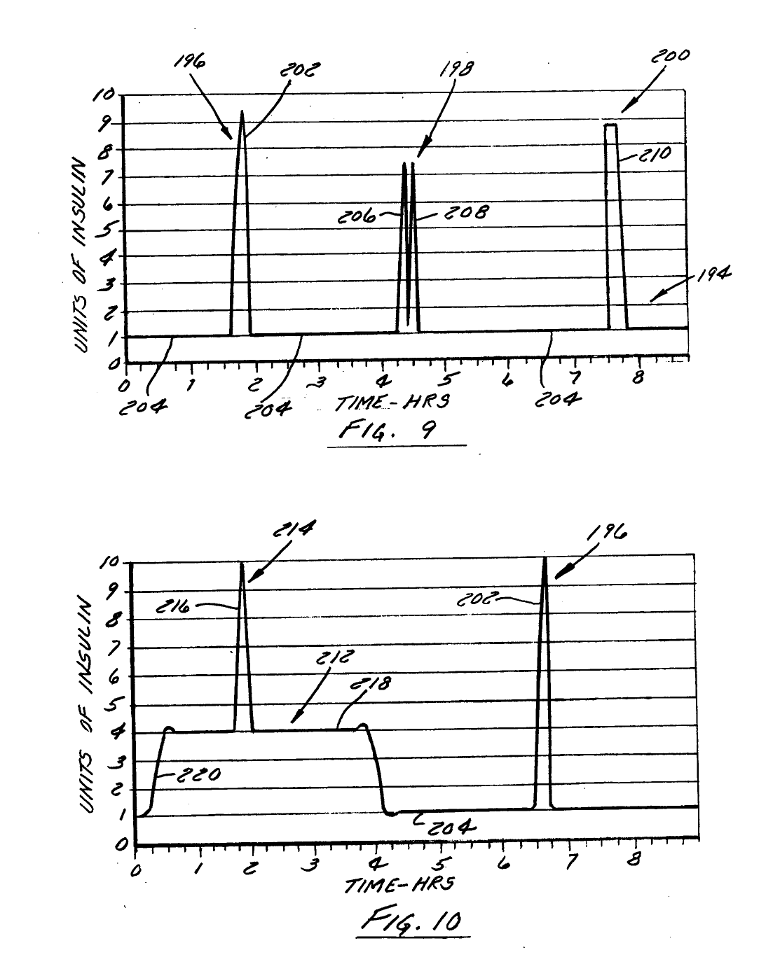

FIG. 9 illustrates a curve 194 depicting three different occlusion prevention and remediation cycles 196, 198 and 200 produced by operating an occlusion prevention and remediation apparatus 26a, 26a′, 26a″ or 26b constructed in accordance with the present invention in a manner in accordance with that discussed above. Each occlusion prevention and remediation cycle 196, 198 or 200 is characterized by a pulse that produces a pressure in the liquid 28, e.g. insulin, within the catheter line 40 that is at least 1.25 times greater than the working pressure of the liquid 28 within the line 40 immediately before beginning the occlusion prevention and remediation cycle. In the occlusion prevention and remediation cycles 196, 198 and 200 shown in FIG. 9, each pulse has a pressure of at least three times the working pressure of the liquid 28 in the catheter line 40 prior to initiation of the cycle.

With continued reference to FIG. 9, occlusion prevention and remediation cycle 196 consists of a single pressure pulse spike 202 that has a pressure of at least 1.25 times working pressure. For example, where the pressure of insulin during basal flow is 1 psig, the pressure pulse spike of the cycle 196 is it least 1.25 psig. For the example depicted in FIG. 9, the single pressure spike 202 has a pressure of at least 1.25 times working pressure and the flow rate of insulin delivery is at least three times the basal rate 204 during at least the peak or apex of the pulse 202.

In a preferred method of carrying out an occlusion prevention and remediation cycle, e.g. cycle 196, the pressure of the pulse spike 202 during the cycle 196 is it least 1.5 times the pressure of the insulin during basal flow 204. In another preferred method of carrying out an occlusion prevention and remediation cycle 196, the pressure of the pulse 202 during the cycle 196 is it least 3 times the pressure of the insulin during basal flow 204. In the curve shown in FIG. 9, the pressure pulse spike 202 of the cycle 196 causes a flow rate to occur during the pulse that is over nine times basal flow 204.

Occlusion prevention and remediation cycle 198 consists of a plurality of pressure spikes 206, 208, each of which increases the pressure of liquid 28 in the catheter 30 to at least 1.25 times the pressure of the liquid 28 in the catheter 30 during basal flow 204 (i.e., working pressure). In a preferred implementation of the occlusion prevention and remediation cycle 198, each one of the pulses 206 and 208 results in the pressure of the liquid 28 inside the catheter line 40 increasing to a pressure that is at least 1.5 times working pressure. In still another preferred implementation, the pressure is increased to at least three times working pressure. While only two pressure spikes 206 and 208 are shown for cycle 198, three or more successive pressure spikes can be employed.

Occlusion prevention and remediation cycle 200 is carried out at similar pressures and flow rates as one or both of the previously discussed cycles 196 and 198 and except that the duration of the pulse is longer so as to form a square wave shaped pulse 210. Of course, other pulse shapes and waveforms are possible.

In a preferred implementation of a method of occlusion prevention and remediation carried out in accordance with the present invention, at least a plurality of cycles are performed during each 24 hour period of time. In one such implementation, a plurality of cycles, each including at least one pressure pulse, are executed during basal flow during each 24 hour time period. In a preferred variation of this implementation, there is at least one hour between cycles and no more than twelve hours between cycles. In one such preferred implementation, there is a plurality of pairs of cycles, e.g., 196, 198 and/or 200, executed during each basal insulin flow delivery cycle.

FIG. 10 illustrates that one or more occlusion prevention and remediation cycles can be carried out even during a bolus insulin flow delivery cycle. As is shown in FIG. 10, a square wave bolus cycle 212 includes a single occlusion prevention and remediation cycle 214 that consists of a single pressure pulse spike 216 that is executed while the bolus cycle 212 has reached a plateau or steady-state condition 218. If desired, the occlusion prevention and remediation cycle 214 can be executed during the ramp up phase 220 of the bolus cycle 212 with the cycle 214 terminating or ending when the bolus plateau 218 is reached. Of course, one or more occlusion prevention and remediation cycles, e.g. cycle 196, can also be executed during a basal insulin flow delivery cycle 204, including in the manner discussed above with regard to FIG. 9.

While the present method and apparatus of the invention can be used with human patients, it is also contemplated that it can be used in the treatment of animals, such as dogs, cats, horses, cows, and the like.

It is also to be understood that, although the foregoing description and drawings describe and illustrate in detail one or more preferred embodiments of the present invention, to those skilled in the art to which the present invention relates the present disclosure will suggest many modifications and constructions as well as widely differing embodiments and applications without thereby departing from the spirit and scope of the claimed invention.

It is claimed:

1. An occlusion prevention or remediation apparatus for a medication delivery device that communicates medication-containing liquid to a catheter line connected to a patient, the occlusion prevention or remediation device comprising a pulse generator that causes a pulse to form in medication-containing liquid in the catheter line downstream of the medication delivery device.

2. The occlusion prevention or remediation apparatus of claim 1 wherein the pulse generator comprises a component separate from the medication delivery device.

3. The occlusion prevention or remediation apparatus of claim 2 wherein the pulse generator is retrofittable or retrofitted onto the medication delivery device.

4. The occlusion prevention or remediation apparatus of claim 1 further comprising a pulse director disposed between the medication delivery device and the pulse generator that directs a pulse formed by the pulse generator away from the medication delivery device.

5. The occlusion prevention or remediation apparatus of claim 4 wherein the pulse director comprises a one-way valve.

6. The occlusion prevention or remediation apparatus of claim 5 wherein the one-way valve comprises a ball-type check valve.

7. The occlusion prevention or remediation apparatus of claim 4 wherein the medication delivery device includes a pump and a discharge from which medication-containing liquid pumped by the pump is discharged to the catheter line and wherein the pulse generator communicates with medication-containing liquid in the catheter line with the pulse director located between the discharge and the pulse generator.

8. The occlusion prevention or remediation apparatus of claim 1 wherein the pulse generator further comprises a drive and a pulse effector in operable communication with the drive and in operable communication with medication-containing liquid in the catheter line.

9. The occlusion prevention or remediation apparatus of claim 8 further comprising an accumulator that stores energy used to operate the pulse effector in forming a pulse in the medication-containing liquid in the catheter line.

10. The occlusion prevention or remediation apparatus of claim 9 wherein the medication delivery device comprises a source of electrical power and the accumulator is coupled to the drive and stores energy from the drive.

11. The occlusion prevention or remediation apparatus of claim 8 wherein the pulse generator comprises a pressure pulse chamber in fluid flow communication with medication-containing liquid in the catheter line and the pulse effector is constructed and arranged to cooperate with liquid in the pressure pulse chamber to form a pressure pulse that is communicated to medication-containing liquid in the catheter line.

12. The occlusion prevention or remediation apparatus of claim 11 wherein the pulse effector comprises a reciprocating element that moves one of a diaphragm and a bladder that is in direct contact with liquid in the pressure pulse chamber.

13. The occlusion prevention or remediation apparatus of claim 12 wherein the liquid in the pressure pulse chamber holds medication-containing liquid that is in direct fluid-flow communication with medication-containing liquid in the catheter line.

14. The occlusion prevention or remediation apparatus of claim 1 wherein the pulse generator comprises (a) a pressure pulse chamber that contains medication-containing liquid that is in fluid flow communication with medication-containing liquid in the catheter line, (b) a drive, and (c) a displaceable pulse effector in operable communication with the medication-containing liquid in the pressure pulse chamber that forms a pulse of increased pressure in medication-containing liquid in the pressure pulse chamber that travels through the medication-containing liquid in the catheter line.

15. The occlusion prevention or remediation apparatus of claim 14 further comprising a one-way fluid valve between the pressure pulse chamber and the medication delivery device.

16. The occlusion prevention or remediation apparatus of claim 15 wherein the medication delivery device comprises an insulin pump, the medication-containing fluid comprises insulin, and the catheter line is connected to an infusion set that extends subcutaneously into tissue of the patient.

17. The occlusion prevention or remediation apparatus of claim 15 wherein the medication delivery device comprises an infusion pump powered by an electrical power source, the drive comprises an electric actuator that is run off the electrical power source of the infusion pump, and the catheter line delivers medication-containing liquid subcutaneously into the patient intravenously or into tissue of the patient.

18. The occlusion prevention or remediation apparatus of claim 17 wherein the electric actuator comprises one of an electric motor, a linear actuator or a rotary actuator.

19. An occlusion prevention or remediation apparatus for a medication delivery device that communicates medication-containing liquid to a catheter line connected to a patient, the occlusion prevention or remediation device comprising:
a pulse generator that causes a pulse to form in medication-containing liquid in the catheter line downstream of the medication delivery device; and
a check valve disposed between the pulse generator and the medication delivery device.

20. The occlusion prevention or remediation apparatus of claim 19 wherein the pulse generator comprises (a) a pressure pulse chamber that contains medication-containing liquid that is in fluid flow communication with medication-containing liquid in the catheter line, (b) a drive, and (c) a displaceable pulse effector in operable communication with the medication-containing liquid in the pressure pulse chamber that forms a pulse of increased pressure in medication-containing liquid in the pressure pulse chamber that travels through the medication-containing liquid in the catheter line.

21. The occlusion prevention or remediation apparatus of claim 20 wherein the medication delivery device is an insulin pump, the medication-containing liquid in the catheter line comprises insulin, the catheter line is connected to an infusion set, and displacement of the displaceable pulse effector causes a pulse in insulin in the catheter line that has a pressure of at least 1.5 times the working pressure of insulin in the catheter line that travels to insulin in the infusion set.

22. The occlusion prevention or remediation apparatus of claim 21 wherein the occlusion or remediation apparatus is integrally formed as part of the insulin pump.

23. The occlusion prevention or remediation apparatus of claim 21 wherein the occlusion or remediation apparatus is constructed and arranged to be retrofit to the insulin pump.

24. An occlusion prevention or remediation apparatus for a medication delivery device that communicates medication-containing liquid to a catheter line connected to a patient, the occlusion prevention or remediation device comprising:
a pulse generator that causes a pulse to form in medication-containing liquid in the catheter line downstream of the medication delivery device that includes a pressure pulse chamber holding medication-containing liquid, a displaceable pulse effector that generates a pulse in medication-containing liquid in the pressure pulse chamber when displaced, a drive, and an accumulator linked to the drive for storing energy from the drive and linked to the pulse effector for displacing the pulse effector using stored energy from the drive; and
a check valve disposed between the pulse generator and the medication delivery device.

25. A method of occlusion prevention or remediation comprising:
(a) introducing a flow of liquid in a line of a catheter infusing a patient; and
(b) perturbing the liquid in the catheter line with a plurality of pressure pulses.

26. The method of claim 25 wherein there is a catheter-tissue interface where the catheter is subcutaneously inserted into the patient and wherein each pressure pulse formed during step (b) reaches the catheter tissue interface proactively preventing occlusion formation.

27. The method of claim 25 wherein there is a catheter-tissue interface where the catheter is subcutaneously inserted into the patient and wherein each pressure pulse formed during step (b) reaches the catheter tissue interface opening up an occlusion in the catheter line or at the catheter-tissue interface.

28. The method of claim 25 comprising (1) a medication delivery device that communicates liquid to the catheter line that conveys the liquid to a catheter-tissue interface of the patient and (2) an occlusion prevention or remediation apparatus disposed between the medication delivery device and a catheter-tissue interface that is in fluid flow communication with liquid in the catheter and that perturbs liquid in the catheter line during step (b).

29. The method of claim 28 further comprising a flow director disposed between the medication delivery device and the occlusion prevention or remediation apparatus and wherein during step (b), the flow director causes each one of the plurality of catheter line liquid pressure pulses generated by the occlusion prevention or remediation apparatus is directed away from the therapeutic or medical liquid delivery device toward the catheter-tissue interface.

30. The method of claim 28 wherein the medication delivery device comprises an insulin pump and the liquid comprises insulin with the pump having a basal insulin flow delivery cycle and a bolus insulin flow delivery cycle wherein the plurality of pulses during step (b) are spaced apart by at least one hour and each one of the plurality of pulses increases the pressure of insulin in the catheter line to at least 1.5 times the working pressure of the insulin in the catheter line.

31. The method of claim 30 wherein during step (b) the plurality of pulses are produced by the occlusion prevention or remediation apparatus during the basal insulin flow delivery cycle.

32. The method of claim 30 wherein during step (b) there are plurality of pulses that are produced by the occlusion prevention or remediation apparatus during each 24 hour operating cycle of the insulin pump.

33. The method of claim 32 wherein during step (b) at least a plurality of pairs of pulses are produced.

34. The method of claim 28 wherein the occlusion prevention or remediation apparatus comprises an accumulator that drives a pulse effector arrangement that operatively communicates with liquid in the catheter line to produce each one of the plurality of pressure pulses during step (b) and wherein before step (b) the accumulator is charged with energy during a period of no or low power medication delivery device operation.

35. The method of claim 34 wherein (1) the medication delivery device comprises an insulin pump that is powered by a battery that discharges insulin from a cartridge within the insulin pump using an electric insulin pump motor powered by the battery with the insulin pump having a basal insulin flow delivery cycle and a bolus insulin flow delivery cycle, and (2) the occlusion prevention or remediation apparatus further comprises an electric motor drive powered by the battery that is used to charge the accumulator during a basal insulin flow delivery cycle.

36. The method of claim 28 wherein the occlusion prevention or remediation apparatus comprises a liquid-holding pulse chamber in fluid flow communication with liquid in the catheter line, a pulse effector in operable cooperation with liquid in the pulse chamber, and a pulse drive that operably cooperates with the pulse effector in generating the plurality of pulses in step (b).

37. The method of claim 36 wherein the pulse drive comprises one of an electric motor and an electromagnetic actuator, the pulse chamber comprises a cavity holding a volume of liquid and that is in fluid flow communication with liquid in the catheter line, and the pulse effector comprises a reciprocable armature.

38. The method of claim 36 wherein the liquid perturbing step (b) comprises the pulse drive causing the pulse effector to be displaced toward the pulse chamber discharging liquid in the pulse chamber into the catheter line in generating each one of the plurality of pressure pulses.

39. The method of claim 38 wherein the pulse chamber comprises a flexible bladder that holds liquid and is in fluid-flow communication with liquid in the catheter line and wherein the pulse effector compresses the bladder during pulse generation when displaced by the pulse drive.

40. The method of claim 36 further comprising an accumulator disposed between the pulse drive and the pulse effector that receives and stores energy inputted from the pulse drive and selectively discharges stored energy to the pulse effector and comprising the additional step before the liquid perturbing step (b) of operating the pulse drive to charge the accumulator and during step (b) discharging the accumulator to drive the pulse effector.
